(19) United States
(12) Patent Application Publication
(10) Pub. No.: US 2013/0289577 A1
(43) Pub. Date: Oct. 31, 2013

(54) Scapuloblade Shoulder Dystocia Device

(71) Applicant: Leah BERHANE, Gaithersburg, MD (US)

(72) Inventor: Leah BERHANE, Gaithersburg, MD (US)

(21) Appl. No.: 13/887,754

(22) Filed: May 6, 2013

Related U.S. Application Data
(63) Continuation of application No. 13/336,602, filed on Dec. 23, 2011, now abandoned.

(51) Int. Cl. A61B 17/44

(52) U.S. Cl. CPC A61B 17/442
USPC 606/123; 606/122

(57) ABSTRACT

An apparatus used for treating shoulder dystocia is provided. The apparatus is a Scapuloblade device with a handle, and a blade section including a first curved side and a flat second side opposite the first side. The curved first side is configured to fit the curvature of a mother's birth canal. The flat side may be bare, may include a friction padding, or may include diaphragm that applies a suction force. The device may be used to rotate the shoulders by applying torque on the scapula region of a baby's anterior shoulder with the flat side of the blade. The elongated handle, which may be an integral continuation of the blade, may facilitate the user to apply a rotational force to dislodge the impacted anterior shoulder so that the shoulders will be aligned with the widest diameter of the inlet of the birth canal of the mother for delivery.

### CROSS REFERENCE TO RELATED APPLICATION AND CLAIM TO PRIORITY

This application is a continuation of prior U.S. Non-Provisional patent application Ser. No. 13/336,602, filed Dec. 23, 2011, entitled “Scapuloblade Shoulder Dystocia Forcep Device,” the disclosure of which is incorporated herein by reference in its entirety.

### FIELD OF THE INVENTION

The present invention relates to a medical device, and in particular, a medical device used in the delivery of a baby that encounters shoulder dystocia. Specifically, the device includes an elongated handle and a blade section with a first side and a second side opposite the first side, the first side being curved and the second side being flat.

### BACKGROUND OF THE INVENTION

Shoulder dystocia is an obstetric emergency that occurs during childbirth, and can be traumatic for the fetus and/or mother, and challenging for the providers. Shoulder dystocia is unpredictable. Shoulder dystocia occurs when, following the delivery of a baby's head, one of the baby's shoulders gets impacted against the pubic arch or symphysis, and fails to deliver with the application of a gentle downward force. A difficult shoulder dystocia can result in injury to the fetus, which ranges from transient neurological deficits due to stretching of the brachial plexus (a large bundle of nerves passing from the neck to the shoulders) to permanent paralysis of the arm due to nerve damage. Other injuries are possible, even brain injury or death caused by lack of oxygen due to an extended delay before the delivery of the baby. Once shoulder dystocia is diagnosed, a series of maneuvers are usually applied including the McRobert's maneuver (flexing the mother's thighs against her abdomen), application of suprapubic pressure (Rubin I maneuver), rotational maneuvers, delivery of the posterior arm or changing the position of the mother to all fours. The rotational maneuvers, which include Rubin II, Wood or cork-screw and reverse Wood screw, are aimed at limiting the stretch on the neck of the baby and hence on the brachial plexus nerve bundles.

This invention provides a method of rotating the shoulders of the baby in the birth canal without applying tension on the brachial nerves so that the baby can be rotated to a position which will facilitate delivery effectively and expeditiously. With the exception of the “baby shoulder vacuum” (U.S. Publication No. 2009/0270879 A1 (Tong)), which aims to apply a vacuum cup directly on the impacted shoulder under the pubic arch to provide traction on the impacted shoulder, there are no other devices to help resolve a shoulder dystocia. The shoulder vacuum is designed to pull on the impacted shoulder by introducing a cup that would fit over the impacted shoulder, which may be difficult due to extreme limitation of space between the impacted shoulder and the pubic arch which caused the dystocia to begin with.

Various obstetric forceps or vacuum devices are available but are not designed to be applied to the baby's shoulders with the exception of Tong's Baby Shoulder Vacuum Cup. The majority of devices are designed for application on or around the baby's head. The baby shoulder cup, would require assembly of different parts and attachment to a vacuum device which could prolong the time to delivery. It may also be difficult to apply the baby cup to the impacted shoulder due to limitation of space between the shoulder and the mother's pubic arch. The manual rotation methods commonly used and described above by inserting two digits behind or in front of the shoulder and pushing can be limited due to the thickness of the fingers and at times and the shortness of the fingers or the limited force that can be generated using just the fingers.

### SUMMARY OF THE INVENTION

The current invention of the Scapuloblade introduces a narrow and streamlined blade which will be slipped to the left to the right side of the impacted shoulder, with a first curved maternal side and a second flattened baby side covered by a diaphragm or padding which will be applied on the scapula region of the baby's shoulders, either the impacted or the opposite shoulder. The device helps rotate the shoulders with no pulling on the neck of the baby or the impacted shoulder.

The Scapuloblade device is a single unit that comprises a streamlined blade that has a first curved maternal side and a second flattened baby side, and is directly inserted into the portion of the birth canal with the most room during a shoulder dystocia. The device can be applied to either side of the impacted shoulder. The handle that extends from the blade as an integral unit can then be grasped by a user and used to apply rotational force to rotate one or the other of the baby's shoulders. The Scapuloblade has a maternal curved outer surface to conform to the birth canal and a flattened surface that can be covered with a diaphragm or padding on the baby's side to assure adherence to or to create a seal on the scapula region of the baby's shoulder. By serving as a lever, the Scapuloblade of the instant invention facilitates, and more effectively allows the rotation, without pulling on the baby that will align the axis of the shoulders in the largest diameter of the birth canal. Additionally, because the device is not designed to apply traction, this further minimizes the risk of injury to the brachial plexus nerves which typically suffer stretch injury.

### BRIEF DESCRIPTION OF THE DRAWINGS

FIG. 1 illustrates a front view of one embodiment of the present invention.

FIG. 2 illustrates a side view of the invention of FIG. 1, the invention being equipped with a padding on the flat surface of the invention.

FIG. 3 illustrates cross section perspective view of the invention of FIG. 1, the invention being equipped with a diaphragm on the flat surface of the invention.

FIG. 4 illustrates a side view of the diaphragm apparatus of FIG. 3.

FIG. 5 illustrates a side view of the baby in the birth canal with the invention of FIG. 1 being applied to the anterior impacted shoulder of the baby in the birth canal after the head is delivered.

FIG. 6 illustrates a side view of the baby's body in the birth canal with the invention of

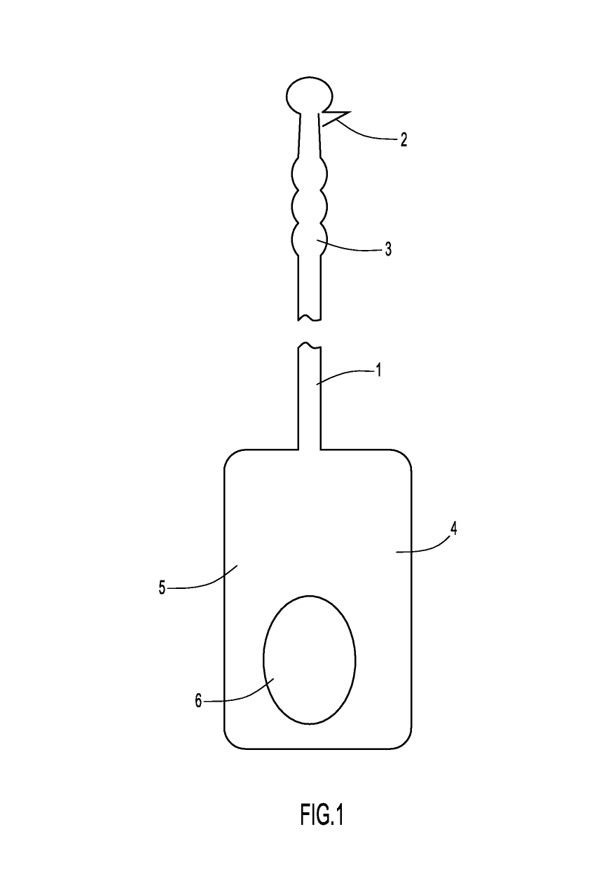

FIG. 1 applied onto the scapula region of a baby's posterior shoulder after the head is delivered.

FIG. 7 illustrates a front view of the baby in the birth canal after the head is delivered with the invention of FIG. 1 applied to the anterior shoulder of the baby.

FIG. 8 illustrates a front view of the baby in the birth canal after the head is delivered with the invention of FIG. 1 applied to the posterior shoulder of a baby.

Like reference numerals have been used to identify like elements throughout this disclosure.

### DETAILED DESCRIPTION OF THE INVENTION

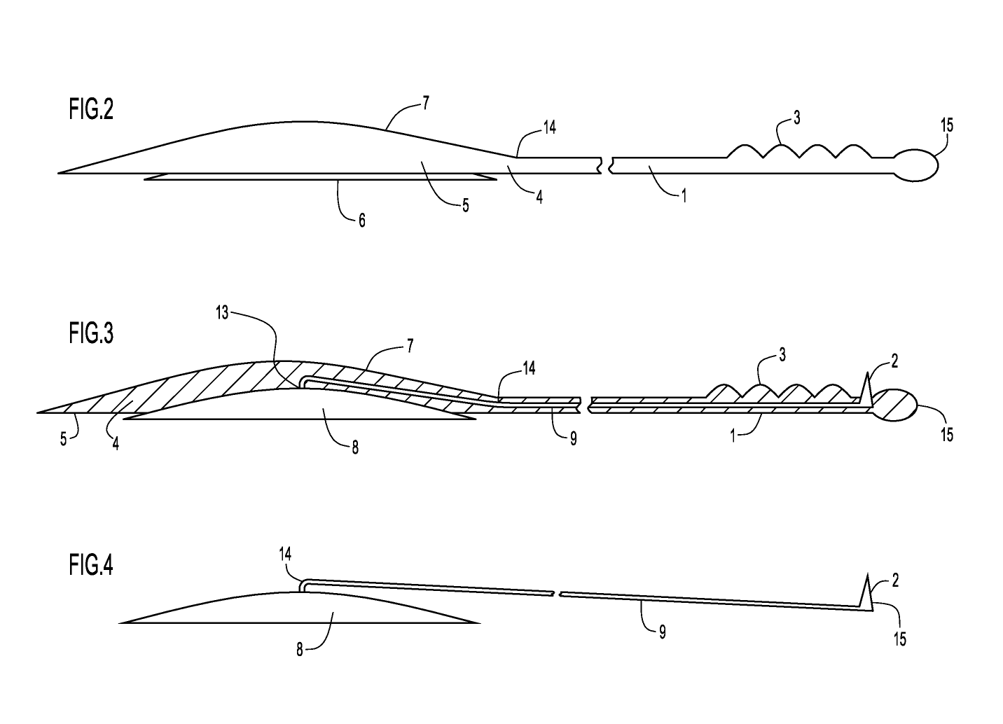

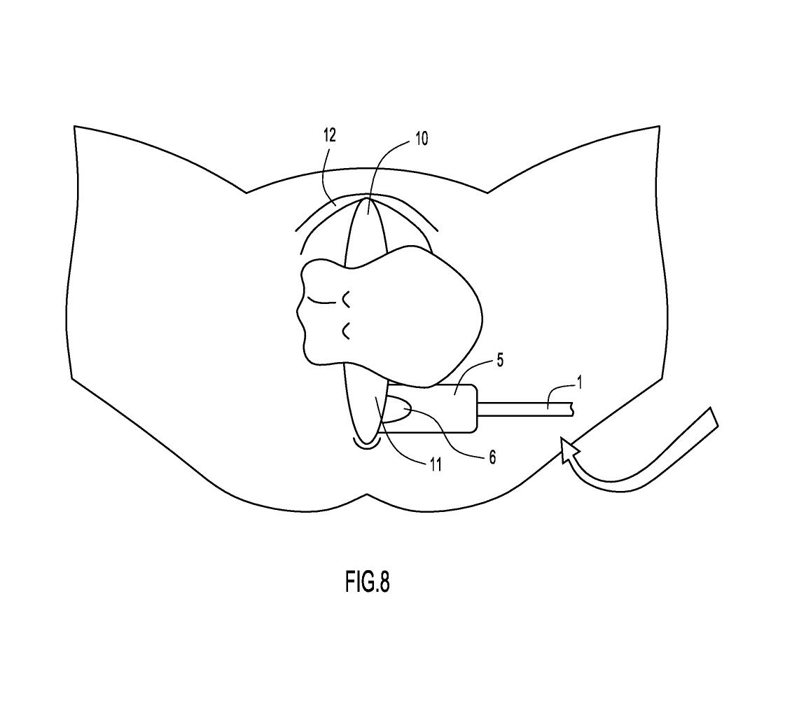

Referring now in detail to the drawings, FIGS. 1-8 illustrate the handle 1 of the

Scapuloblade device. As illustrated in FIGS. 1-8, the handle 1 may be elongated with a distal end 14 and a proximal end 15. Disposed on the distal end 14 of the handle 1 is a blade 4. The handle 1 may be constructed of the same material as the blade 4. Furthermore, the handle 1 may be continuous with the blade 4, and can be of different angulations with the blade 4, and of different lengths and thicknesses. The blade 4 of the Scapuloblade can be oblong and rectangular, oval or more circular or a square with rounded angles in its shape, and can vary in thickness to fit different space limitations between the baby and the mother's birth canal. However, a blade 4 with a narrow rectangular shape with rounded angles will combine depth, optimal area of contact with baby's skin, and likelihood of fitting in the tight space between the baby and the mother's birth canal but not protruding beyond so as to limit risk of injury to mother's vagina. The blade 4 may be constructed from metal or a polymeric material.

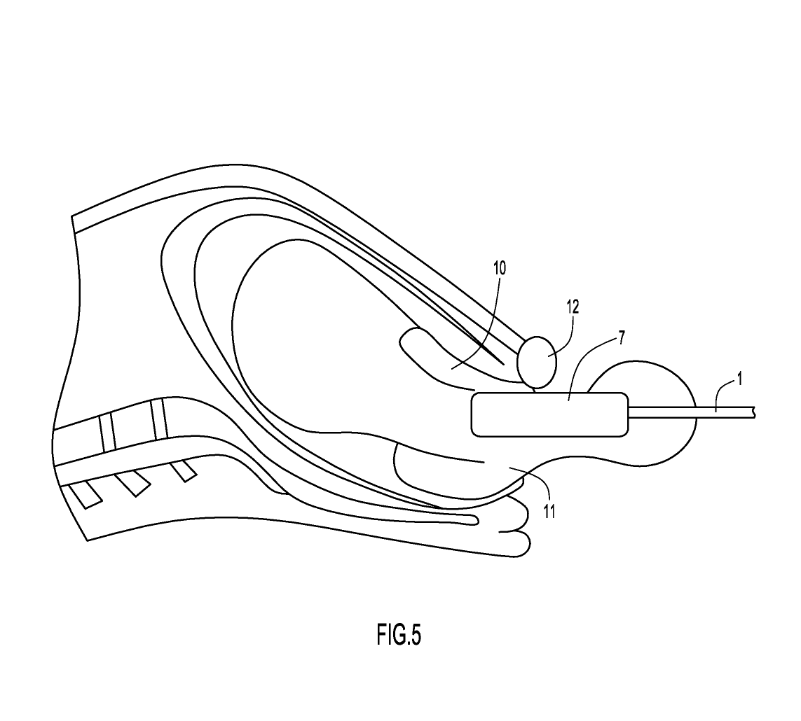

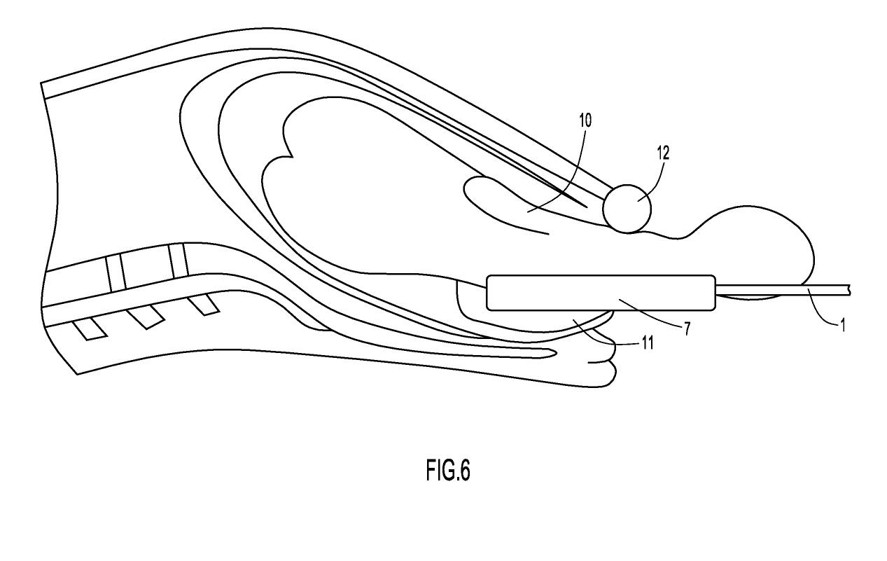

FIGS. 1, 2, and 3 illustrate the scalloping 3 on the handle 1 of the Scapuloblade to allow firm grasp to the user. Additionally, this scalloping 3 may or may not be overlaid with padding of polymer material to allow a firm grasp by minimizing slippage. Illustrated in FIGS. 1, 2, 7 and 8, a padding 6 of polymer material is disposed and overlying the solid flat part 5 of the blade 4. The padding 6 enhances the contact with the baby's skin by friction. In other embodiments, the flat side 5 of the blade 4 may also be bare without padding 6. FIGS. 1, 2, 3, 5, and 6 illustrates the curved side 7 of the blade 4 to conform to the curvature of the mother's birth canal. FIGS. 3 and 4 illustrate the optional diaphragm 8 disposed on the blade 4. The diaphragm 8 may be disposed through an opening 13 on the flat part 5 of the blade 4. The diaphragm 8 may enhance contact with baby's skin by creating a suction force on the shoulder region of the baby. Attached and in fluid communication with the diaphragm 8 is a tube 9. The tube 9 may be connected to a lever 2 that may be disposed on the handle 1 of the Scapuloblade. When the lever 2 is pushed or deflected on the handle 1, the force will be transmitted through tube 9 which will cause a depression of the diaphragm 8 releasing the air inside the diaphragm 8, and causing a suction attachment of the diaphragm 8 to the skin of the baby. The lever 2 can be used to activate and release the suction force of the diaphragm 8. Alternately, the diaphragm 8 can be activated by pushing it against the baby's upper back to create suction effect and the lever 2 used to release the suction after the rotation of the baby.

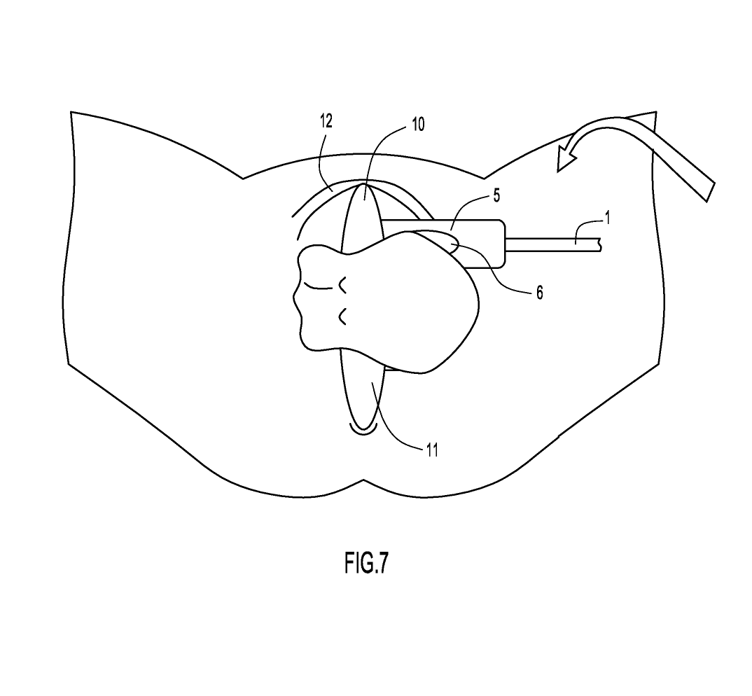

As illustrated in FIGS. 5-8, a Scapulobalde can be used when shoulder dystocia is diagnosed, with impaction of the anterior shoulder 10 behind the mother's pubic symphysis 12. The Scapuloblade may be packaged sterilely as a single unit, and stored in the birth room. The user will insert the blade 4 of the Scapuloblade while holding onto the handle 1. When the blade 4, is inserted into the birth canal, the curved side 7 of the blade 4 is placed against the mother's vagina and the flat side 5 of the blade 4 is placed against the baby's upper back. The proper placement of the blade 4 will be guided by the user, to assure that the padding 6, diaphragm 8, or bare flat side 5 of the blade 4 is placed against the scapula region of the baby's, impacted anterior shoulder 9, as best illustrated in FIGS. 5 and 7. Once the correct placement and depth is assured, if the Scapuloblade has a diaphragm 8, the user can press the blade 4 against the baby's anterior shoulder 10, or can push the lever 2 on the handle 1 to activate, and later release, the suction force of the diaphragm 8 against the baby's skin. Alternatively, the presence of the padding 6 can be sufficient to create enough friction against the baby's skin to avoid slippage. Because of the narrowness of the space between the baby's anterior shoulder 9 and the mother's birth canal, a bare blade 4 against the baby's skin may also achieve sufficient contact to allow the desired rotation.

With the user's fingers nested between the scalloping 3 of the handle 1, and while stabilizing the Scapuloblade with the other hand on the non scalloping part of the handle 1 or a portion of the curved side 7 of the blade 4, the user can apply torque force on the Scapuloblade, in a curved arc aimed towards the front of the baby. This motion serves to dislodge the anterior shoulder 10 from under the pubic symphysis 12 so that the axis of the shoulders will be rendered to lie in an oblique diameter of the mother's vagina (instead of the original anterior to posterior diameter), which provides the maximal space for the delivery of the shoulders. Once the rotation is accomplished, the user can remove the Scapuloblade and continue the delivery of the baby in the normal manner.

If, on the other hand, difficulty is encountered rotating a deeply impacted anterior shoulder 10, then the blade 4 can be applied to the scapula region of the posterior shoulder 11, as illustrated in FIGS. 5-8. The application of the blade 4 to the posterior shoulder 11 is done in the same manner as described above for the anterior shoulder 10, activating the suction feature if the blade 4 is equipped with a diaphragm 8. The user will then rotate the posterior shoulder 11 until the axis of the shoulders 10, 11 is aligned in the oblique diameter. Once this is achieved, the Scapuloblade can be removed and the delivery completed in the normal fashion or by sweeping the posterior arm forward and out to provide more room for the delivery of the rest of the baby. Rotation of the shoulders 10, 11 will more effectively dislodge an impacted shoulder and will minimize or remove the need for traction on the baby's head, thus minimizing traction injury to the brachial nerve. Rotating the shoulders 10, 11 in an expeditious manner can also minimize the time it takes to deliver the baby and reduce the risk of birth asphyxia. Because this device is a single unit with the handle 1 and blade 4 being integral parts, the Scapuloblade can be manufactured and sold without requiring assembly by the user. Use of the Scapuloblade could also add to the time efficiency of delivering a baby diagnosed with shoulder dystocia. Furthermore, use of the Scapuloblade can replace other maneuvers used to reduce or resolve a shoulder dystocia emergency and can assure efficiency, thus shortening the time of delivery of the baby. Once the success of the Scapuloblade is clinically proven, it can also reduce user anxiety.

It is to be understood that terms such as “left,” “right,” “top,” “bottom,” “front,” “rear,” “side,” “height,” “length,” “width,” “upper,” “lower,” “interior,” “exterior,” “inner,” “outer” and the like as may be used herein, merely describe points or portions of reference and do not limit the present invention to any particular orientation or configuration. Further, terms such as “first,” “second,” “third,” etc., merely identify one of a number of portions, components and/or points of reference as disclosed herein, and do not limit the present invention to any particular configuration or orientation.

Therefore, although the disclosed inventions are illustrated and described herein as embodied in one or more specific examples, it is nevertheless not intended to be limited to the details shown, since various modifications and structural changes may be made therein without departing from the scope of the inventions. Further, various features from one of the embodiments may be incorporated into another of the embodiments. Accordingly, it is appropriate that the invention be construed broadly and in a manner consistent with the scope of the disclosure.

What is claimed is:

1. A device for aiding in the delivery of a baby, comprising:
an elongated handle with a distal end and a proximal end;
a blade disposed on the distal end of the handle, the blade including a substantially curved first side configured to fit the curvature of the mother's birth canal and a substantially flat second side configured to be applied to a shoulder region of a baby, the second side being opposite the first side; and
a padding disposed on the flat second side of the blade, wherein the padding on the second side is configured to frictionally engage a shoulder of a baby within a mother's birth canal.

2. The device of claim 1, further comprising scalloping on the proximal end of the handle.

3. The device of claim 1, further comprising padding disposed on the proximal end of the handle.

4. The device of claim 1, wherein the handle and the blade are constructed from a metallic material.

5. The device of claim 1, wherein the handle and the blade are constructed from a polymeric material.

6. The device of claim 1, wherein the padding disposed on the flat second side of the blade is constructed from a polymeric material.

7. The device of claim 1, wherein the blade is of a substantially rectangular shape.

8. A device for aiding in the delivery of a baby, comprising:
an elongated handle with a distal end and a proximal end;
a blade disposed on the distal end of the handle, the blade including a substantially curved first side and a substantially flat second side, the second side being opposite the first side; and
a diaphragm disposed on the flat second side of the blade, wherein the diaphragm is configured to engage a shoulder region of a baby within a mother's birth canal via a suction force.

9. The device of claim 8, further comprising scalloping on the proximal end of the handle.

10. The device of claim 8, further comprising padding disposed on the proximal end of the handle.

11. The device of claim 8, wherein the handle and the blade are constructed from a metallic material.

12. The device of claim 8, wherein the handle and the blade are constructed from a polymeric material.

13. The device of claim 8, wherein pressing the diaphragm against the shoulder of the baby creates a suction force on the shoulder region of the baby, attaching the device to the shoulder region of the baby.

14. The device of claims 8, wherein the curved first side has a curvature that is configured to fit the curvature of the mother's birth canal.

15. The device of claim 8, wherein the flat second side is configured to be applied to the shoulder region of the baby.

16. A device for aiding in the delivery of a baby, comprising:
an elongated handle with a distal end and a proximal end;
a blade disposed on the distal end of the handle, the blade further comprising:
a first side that is substantially curved, and
a second side opposite the first side that is substantially flat;
an actuator disposed on the proximal end of the handle;
a diaphragm disposed on the second side of the blade, the diaphragm configured to engage a shoulder region of a baby within a mother's birth canal; and
a tube operatively coupling the diaphragm and the actuator so that the actuator and the diaphragm are in fluid communication, wherein actuation of the actuator enables the diaphragm to apply a suction force on the shoulder region of a baby within a mother's birth canal.

17. The device of claim 16, wherein the actuator is capable of activating and releasing the suction force of the diaphragm.

18. The device of claim 16, wherein the actuator is a lever.

19. The device of claim 16, wherein the second side of the blade further comprises an opening disposed in the same location as diaphragm.

20. The device of claim 19, wherein the tube is inserted through the opening to be coupled to the diaphragm.
